(19) United States
(12) Patent Application Publication
(10) Pub. No.: US 2026/0157754 A1
(43) Pub. Date: Jun. 11, 2026

(54) IMPLANTABLE ADJUNCT HAVING A SELECTIVELY LAMINATED FILM ATTACHMENT

(71) Applicant: CILAG GMBH INTERNATIONAL, ZUG (CH)

(72) Inventors: MARK ZEINER, LOVELAND, OH (US); SARAH A. SCULLY, CINCINNATI, OH (US); LAURA S. DOWNING, CINCINNATI, OH (US); RICHARD L. LEIMBACH, CINCINNATI, OH (US); JOHN KIMSEY, CINCINNATI, OH (US)

(73) Assignee: CILAG GMBH INTERNATIONAL, ZUG (CH)

(21) Appl. No.: 19/182,063

(22) Filed: Apr. 17, 2025

Related U.S. Application Data
(60) Provisional application No. 63/649,626, filed on May 20, 2024.

(51) Int. Cl. A61B 17/072; A61B 17/00

(52) U.S. Cl. CPC A61B 17/07207; A61B 2017/00526

(57) ABSTRACT

Systems, apparatuses, and methods for attaching film layers to implantable adjuncts using selective lamination are disclosed. The systems include a first film layer contacting the deck-facing surface. The first film layer includes a first exterior side facing away from the implantable adjunct. The first film layer is selectively heat laminated to the deck-facing surface such that there are laminated portions and unlaminated portions of the first film layer.

### CROSS-REFERENCE TO RELATED APPLICATIONS

This application claims the benefit of U.S. Provisional Application No. 63/649,626, filed on May 20, 2024, the entirety of which is incorporated herein by reference.

### FIELD OF INVENTION

The present disclosure generally relates to implantable adjuncts for surgical staplers. More specifically, the present disclosure relates to implantable adjusting having an attached film layer using selective lamination and methods of attaching a film layer to implantable adjuncts for surgical staplers using selective lamination.

### BACKGROUND

Stapling is a crucial aspect of many surgical procedures, such as gastrointestinal, thoracic, and gynecological surgeries. A pivotal aspect of a stapling procedure is to provide proper staple formation (i.e., the legs curling around at a proper position to secure tissue within the formed staple). Issues can occur, therefore, if conditions exist that take away from the possibility of proper tissue fastening. One such condition is when there is variation in tissue thickness across the length of the stapling surface.

Certain staple cartridges used in stapling procedures may include an implantable adjunct on the deck of the cartridge or on the anvil side of the device. This implantable adjunct can be used to compensate for differences in tissue thickness. For instance, tissue may be thicker at one end of a staple cartridge than at another, yet the length of staple legs is the same for all staples in the cartridge. As such, a staple can have proper length in one section of that tissue yet be too long for another section of the tissue. The implantable adjunct is stapled to the tissue, thereby compensating for some of the thickness variation, and, in some implementations, the implantable adjunct will biodegrade over time.

Since the adjunct is stapled to the tissue, the implantable adjunct must be constructed such that crowns of the staples do not pull through the adjunct—if staples pull through, then the tissue thickness is no longer compensated for. Various designs have been implemented to decrease the chance of staple pull-through. One such method is to include a film layer on the adjunct such that the adjunct comprises multiple layers, including for instance a foam or other porous material that is then laminated with a layer of film material. The multiple layers can include several layers of film and fabric located on either side of the device. In prior implementations, the film layer is heat bonded to the porous layer.

During the heat bonding process, the film material is melted onto the implantable adjunct to cause the film to adhere to the adjunct. Several issues can arise from this heat lamination. For instance, the application of heat during the lamination process can result in the formation of air pockets within the cushion that impact the structural integrity of the implantable adjunct. Heat lamination can also affect the thickness of the implantable adjunct. There is a need for providing an implantable adjunct that includes one or more film attachments and methods for assembling such an implantable adjunct that maintain the structural integrity and performance of the implantable adjunct.

### SUMMARY

It is an object of the present designs to provide devices and methods to meet the above-stated needs. The designs can be for an implantable adjunct having one or more film layers selectively laminated thereto. It is to be understood that the one or more film layers may include various materials, including by way of example, mesh materials, which are applied to the implantable adjunct. The selective lamination in patterns limits the impact to the structural integrity of the implantable adjunct that typically occurs during heat lamination and provides improved performance for the implantable adjunct. Further, the unlaminated areas allow the implantable adjunct to flex with respect to the film layer to prevent film tearing or fracturing around the staple crowns, and thus reduce staple pull-through.

Other aspects of the present disclosure will become apparent upon reviewing the following detailed description in conjunction with the accompanying figures. Additional features or manufacturing and use steps can be included as would be appreciated and understood by a person of ordinary skill in the art.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation. It is expected that those of skill in the art can conceive of and combine elements from multiple figures to better suit the needs of the user.

FIG. 1 is an exploded perspective view of a replaceable staple cartridge without an adjunct.

FIG. 2A is a schematic view showing a replaceable staple cartridge, an attachment material, and an implantable adjunct.

FIG. 2B is a schematic view showing the replaceable staple cartridge, attachment material, and implantable adjunct of FIG. 2A assembled.

FIG. 2C is a perspective view showing the replaceable staple cartridge and implantable adjunct of FIG. 2A assembled.

FIG. 3A is a side-view schematic of a staple cartridge being loaded into a surgical instrument, according to aspects of the present disclosure.

FIG. 3B is a schematic view of an implantable adjunct stapled to tissue, according to aspects of the present disclosure.

FIG. 4A is an exploded view of an implantable adjunct with a film layer and a lamination pattern, according to aspects of the present disclosure.

FIG. 4B is a perspective view of the implantable adjunct in FIG. 4A as assembled, according to aspects of the present disclosure.

FIG. 4C is an exploded view of an implantable adjunct with two film layers and lamination patterns for each layer, according to aspects of the present disclosure.

FIG. 5A is an exploded view of an implantable adjunct with a film layer and a lamination pattern, according to aspects of the present disclosure.

FIG. 5B is a perspective view of the implantable adjunct in FIG. 5A as assembled, according to aspects of the present disclosure.

FIG. 5C is a perspective view of an implantable adjunct similar to the adjunct shown in FIG. 5B, but with the addition of a groove along the longitudinal axis of the adjunct, according to aspects of the present disclosure.

FIG. 6A is an exploded view of an implantable adjunct with two wings provided for side lamination, according to aspects of the present disclosure.

FIG. 6B is a perspective view of the implantable adjunct in FIG. 6A as assembled, according to aspects of the present disclosure.

FIG. 7A is an exploded view of a system comprising an implantable adjunct with a mesh film layer and a corresponding lamination block, according to aspects of the present disclosure.

FIG. 7B is a partially exploded view of the system of FIG. 7A wherein the film layer is attached to the implantable adjunct, according to aspects of the present disclosure.

FIG. 7C is a detail view of the lamination surface of the lamination block in FIG. 7A, according to aspects of the present disclosure.

FIG. 7D is a detail view of the laminated implantable adjunct in FIG. 7B, according to aspects of the present disclosure.

FIG. 7E is cross-sectional view of the system of FIG. 7A being used to laminate the mesh film layer comprising fiber strands to the adjunct using the lamination block, according to aspects of the present disclosure.

FIG. 7F is cross-sectional view of the system of FIG. 7A being used to laminate the mesh film layer comprising fiber strands with flattened profiles to the adjunct using the lamination block, according to aspects of the present disclosure.

FIG. 8A is an exploded view of a system comprising an implantable adjunct with a film layer and a corresponding lamination block, according to aspects of the present disclosure.

FIG. 8B is a partially exploded view of the system of FIG. 8A wherein the film layer is laminated to the implantable adjunct in a predefined pattern, according to aspects of the present disclosure.

FIG. 8C is a detail view of the lamination surface of the lamination block in FIG. 8A, according to aspects of the present disclosure.

FIG. 8D is a detail view of the film layer in FIG. 8A laminated to the implantable adjunct in a pattern defined by the lamination block, according to aspects of the present disclosure.

FIG. 8E is cross-sectional view of the system of FIG. 8A being used to laminate the film layer using the lamination block, according to aspects of the present disclosure.

FIG. 9 is a flowchart for a method for assembling an implantable adjunct, according to aspects of the present disclosure.

### DETAILED DESCRIPTION

Specific examples of the present invention are now described in detail with reference to the Figures, where identical reference numbers indicate elements which are functionally similar or identical. The examples provide solutions for staple cartridge systems that include an implantable adjunct. An implantable adjunct can be used in stapling surgery to account for differing tissue thicknesses across the length of the stapling surface. For instance, a length of tissue clamped in an end effector of a surgical instrument may be thicker at one end of the staple cartridge than at the other end. However, the staple cartridge may be loaded with staples of a single length, meaning the staples may be properly sized for the thicker section of tissue, but may be too long for the thinner section of tissue. If the staples are too long, proper compression of the tissue at the staple site may not be optimal. An implantable adjunct can account for this differing tissue thickness by providing support for the thinner sections of tissue. Where the tissue is thick, the implantable adjunct can be compressed all the way down since no additional thickness is needed to account for the staple length. Where the tissue is thin, the implantable adjunct is not as compressed, meaning the adjunct provides the additional thickness needed to account for the staple length, thereby providing proper compression in that section of the tissue.

The staple cartridge can also include an implantable adjunct. The implantable adjunct is configured to be captured within a staple along with tissue when the staple is deployed by the corresponding driver. The implantable adjunct can comprise a buttress, a tissue thickness compensator, and/or other adjunct material. A tissue thickness compensator is configured to compensate for variations in tissue properties, such as variations in the thickness of tissue, for example, along a staple line. A tissue thickness compensator can be compressible and resilient. In use, a tissue thickness compensator prevents or limits the over-compression of stapled tissue while facilitating adequate tissue compression within and between staples.

The implantable adjunct of a staple cartridge can be releasably secured to the body of the staple cartridge. For example, the implantable adjunct can be releasably secured to the deck of the staple cartridge with a releasable adhesive, at least one attachment tab, and/or other attachment features.

As discussed above, the implantable adjunct accounts for differing tissue thickness by providing support for the thinner sections of tissue. In that regard, implantable adjunct may include a foam or cushion material that allows for compression. The foam can include one or more film layers attached thereto using a connector, such as a thread. For example, a film layer may be applied to allow the implantable adjunct to more easily slide over tissue by providing a smoother surface than the underlying foam. In other examples, a film layer may be applied to facilitate attachment of the implantable adjunct to the staple deck. It is to be understood that the term film layers could include materials such as meshes or knit fabrics. The attachment of film layers using selective lamination reduces damage to the layers that results from heat bonding to provide additional strength to the implantable layer and improve staple pull through during operation. Further, the selective lamination allows for increased flexibility of the implantable adjunct.

The invention is not necessarily limited to the examples described, which can be varied in construction and detail. The terms “distal” and “proximal” are used throughout this description and are meant to refer to positions and directions relative to the handle of surgical instrument 200. As such, “distal” or distally” refer to a position distant to or a direction away from the handle of surgical instrument 200 (i.e., a direction toward a patient). Similarly, “proximal” or “proximally” refer to a position near or a direction towards the handle of surgical instrument 200 (i.e., toward an operator of the handle). Furthermore, the singular forms “a,” “an,” and “the” include plural referents unless the context clearly dictates otherwise. Furthermore, the use of “couple”, “coupled”, or similar phrases should not be construed as being limited to a certain number of components or a particular order of components unless the context clearly dictates otherwise.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g., “about 90%” may refer to the range of values from 71% to 109%.

The components described herein can be formed from biocompatible materials using manufacturing methods known to those of skill in the art. For example, and not limitation, the components described herein can be formed of a thermoset (e.g., the adjunct) or molded from a thermoplastic (e.g., the film or knit layers).

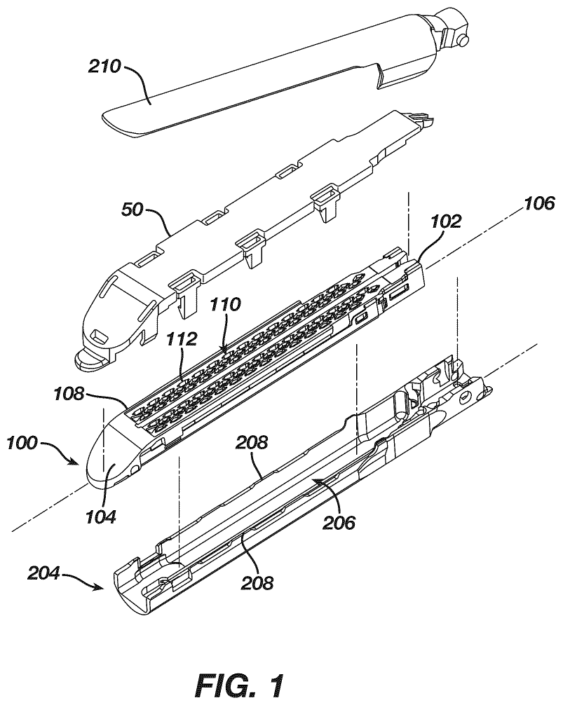

FIG. 1 provides background on how the presently described adjuncts and systems interact with a staple cartridge. FIG. 1 shows an exploded view of a staple cartridge 100 that does not include an implantable adjunct on deck 108 thereof. In these prior examples, retainer 50 can be attached to staple cartridge 100 from proximal end 102 to distal end 104 to ensure that staples within various staple pockets 110 do not fall out of openings 112 within deck 108. Retainer 50, therefore, is a static device with a function of preventing staples from falling out before staple cartridge 100 is positioned within channel 206 of first jaw frame 204 of end effector 202. Retainer 50 can be removed when staple cartridge 100 is inserted between channel rails 208 of channel 206.

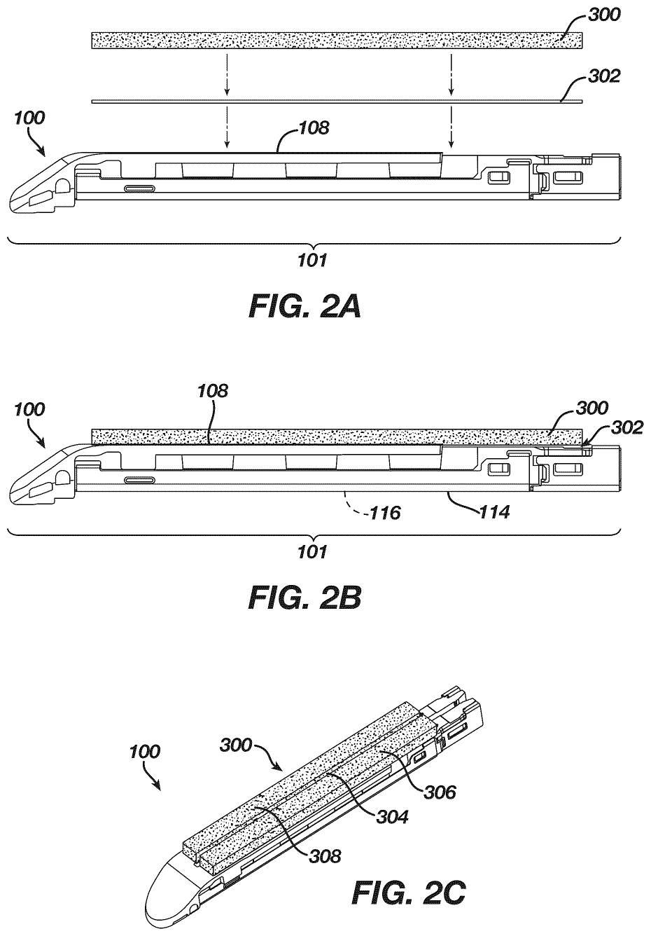

FIGS. 2A-2C illustrate staple cartridges that include an implantable adjunct 300. As seen in the exploded view of FIG. 2A, a system benefiting from the presently disclosed designs includes staple cartridge 100 (which is substantially similar to the staple cartridge shown in FIG. 1) and implantable adjunct 300 which is adhered to deck 108 that is positioned along elongate body 101 of staple cartridge 100. Implantable adjunct 300 can be adhered to staple cartridge 100 with attachment material 302. Attachment material 302 can provide sufficient adhesion for implantable adjunct 300 to remain adhered to deck 108 when being positioned at the treatment site, but the adhesion does not impair the ability of implantable adjunct 300 from being detached from deck 108 when being implanted. In some instances, attachment material 302 can be an adhesive, adhesive strip, double-sided tape, and the like. The attachment material 302 can be or include a pressure sensitive adhesive such that applying pressure to implantable adjunct 300 causes the attachment material 302 to be compressed and increases the adhesion of the attachment material 302 to the cartridge 100 and the implantable adjunct 300. In other words, compression of the adjunct 300 against the cartridge 100 can increase the adhesion of the implantable adjunct 300 to the cartridge 100 via the attachment material 302. The compression of the attachment material 302 can be less than a second and greatly increases the adhesion of the adjunct 300 to the cartridge.

FIG. 2B shows implantable adjunct 300 adhered to deck 108 via attachment material 302. FIG. 2C is a perspective view of implantable adjunct 300 adhered to deck 108. For background, the staples of the systems described herein are fired through implantable adjunct 300 during the stapling procedure. In some instances, implantable adjunct 300 can include sled groove 304 along the longitudinal axis 350 of the adjunct. Sled groove 304 provides a path for a knife (not shown in figures) to traverse such that the knife does not need to cut through adjunct 300, thereby preserving the edge on the knife. When implantable adjunct 300 includes sled groove 304, implantable adjunct 300 can be considered to be separated into adjunct first side 306 and adjunct second side 308. In some examples, implantable adjunct 300 can include laminated layers, such as a foam and/or porous material laminated with a mesh material, wherein the sled groove 304 is disposed in the foam and/or porous material but the mesh material remains intact. In other examples, implantable adjunct 300 can include a film layer and/or a mesh layer and/or a knit fabric layer, as will be described in greater detail below.

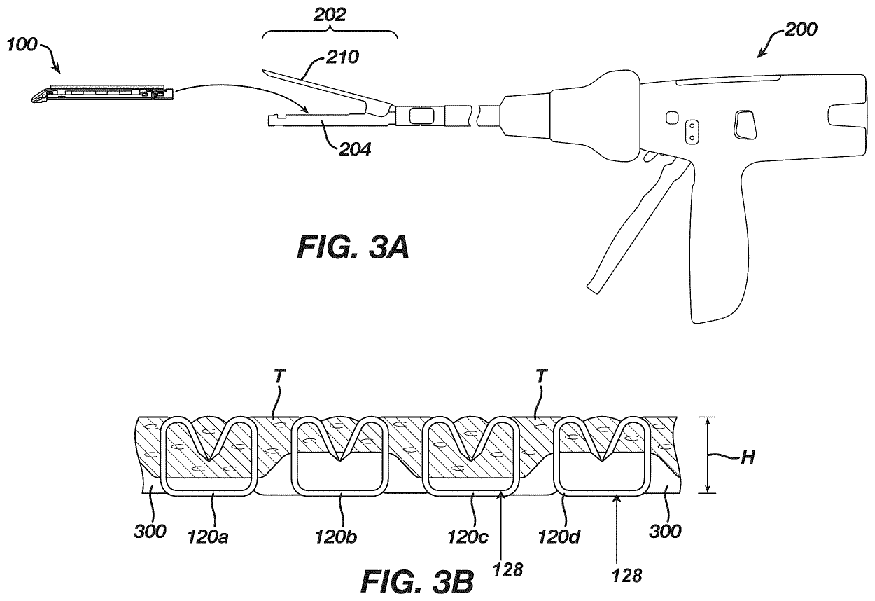

FIG. 3A is a side-view schematic of staple cartridge 100 being loaded into a surgical instrument, i.e., surgical instrument 200. Staple cartridge 100 is loaded into end effector 202 before being positioned at the treatment site. As described above, staple cartridge 100 is inserted into first jaw frame 204. Anvil 210 clamps down toward staple cartridge 100 during the stapling procedure. Once the tissue is stapled, anvil 210 opens to leave the staples and implantable adjunct 300 attached to the tissue. Staple cartridge 100 remains in first jaw frame 204 as surgical instrument 200 is removed from the treatment site. Although FIG. 3A shows staple cartridge 100 without a retainer attached thereto, some example retainers described herein can be configured to be inserted into first jaw frame 204 while attached to the staple cartridge 100. As is shown in FIG. 2B, cartridge 100 includes a pan 114 to support the elongate body 101, the bottom surface 117 of pan 114 being adjacent first jaw frame 204 when inserted into the first jaw frame 204 of end effector 202.

As stated above, implantable adjunct 300 can account for this differing tissue thickness by providing support for the thinner sections of tissue. Where the tissue is thick, implantable adjunct 300 can be compressed all the way down since no additional thickness is needed to account for the staple length. Where the tissue is thin, the implantable adjunct 300 is not as compressed, meaning the adjunct provides the additional thickness needed to account for the staple length, thereby providing proper compression in that section of the tissue. FIG. 3B is a schematic showing the implantable adjunct 300 stapled to tissue (T) having different thickness. The individual staples 120a, b, c, d have the same height (H), so the implantable adjunct 300 fills in the space for thinner sections of tissue (i.e., the tissue (T) shown at staples 120b and 120d). For thicker sections of tissue (i.e., the tissue (T) shown at staples 120a and 120c), the implantable adjunct 300 is more compressed as the staples do not need the additional space (i.e., height) filled in by implantable adjunct 300.

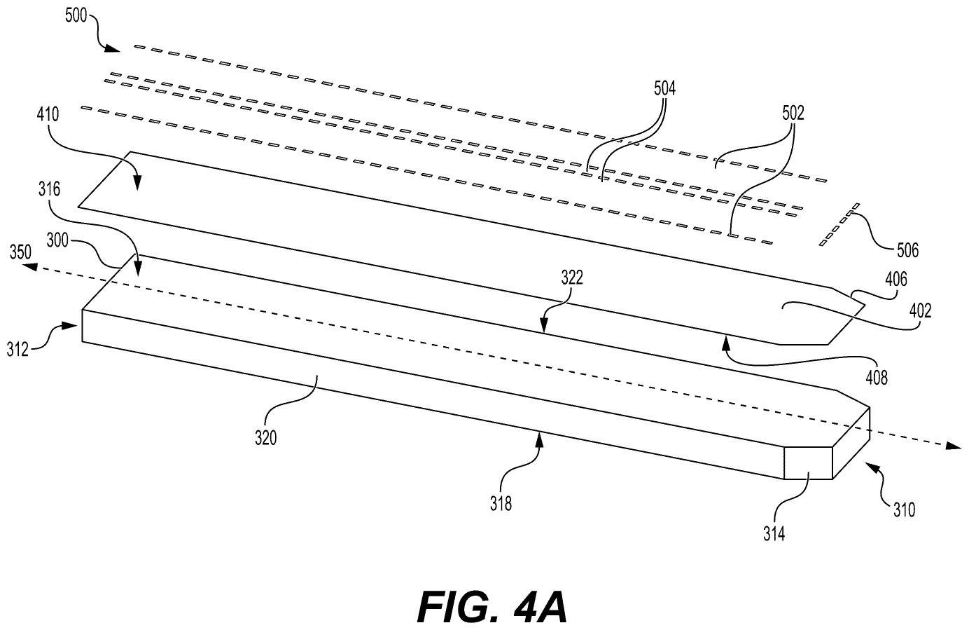

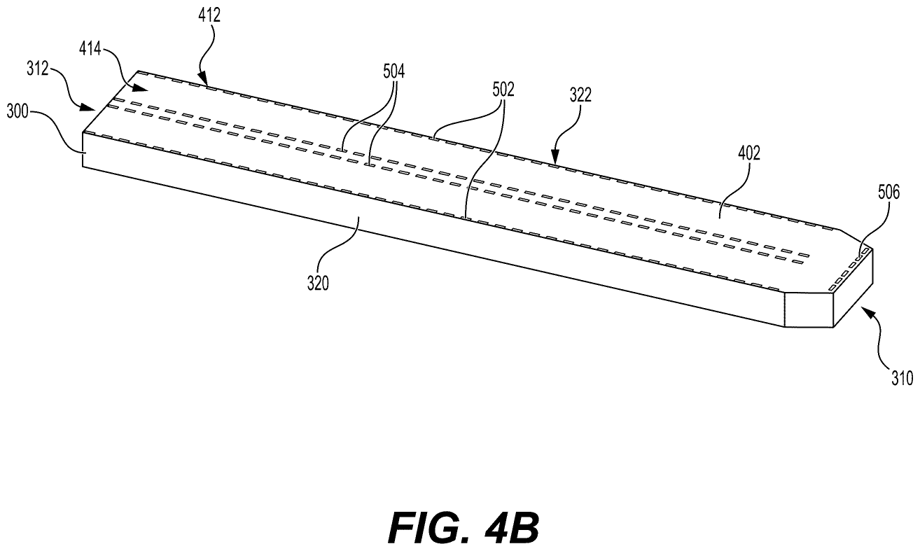

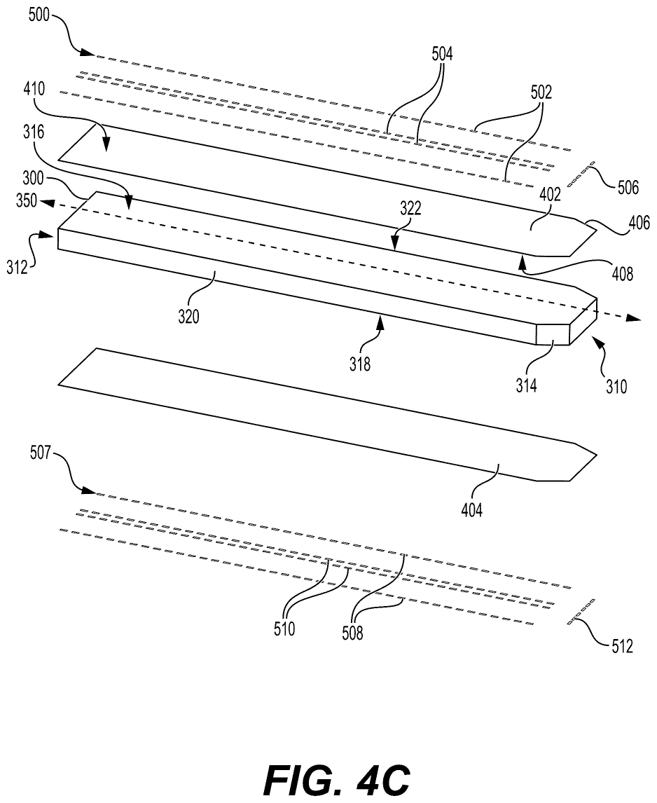

FIGS. 4A and 4B are an exploded view and an assembled view, respectively, of an exemplary implantable adjunct 300 configured to be detachably adhered to deck 108 of staple cartridge 100. Implantable adjunct 300 can be employed in the system described above with respect to FIGS. 2A-2C, for example. Implantable adjunct 300 includes a film layer 402 selectively heat laminated thereto using surface laminations 500 formed in a lamination pattern, as described in further detail below. The pattern of surface laminations 500 is formed by portions of film layer 402 that are heat melted to implantable adjunct 300 using known heat lamination processes in order to attach film layer 402 to implantable adjunct. As shown in FIG. 4C, which is discussed in further detail below, a second film layer 404 can further be selectively laminated to the opposite side of implantable adjunct 300 using surface laminations 507 formed in a lamination pattern. It is to be understood that second film layer 404 could optionally be applied to any of the examples set forth herein. Utilizing selective lamination to attach first film layer 402 and/or second film layer 404 to implantable adjunct 300 advantageously provides for enough connection to hold the film layers securely in place on the adjunct for tissue manipulation and staple firing, while limiting the lamination to specific locations on the adjunct. Limiting the lamination locations limits the structural degradation to the adjunct that is a result of the heat lamination process. The selective lamination thus provides increased structural integrity and improved staple pull through during use in comparison to an adjunct having a film layer laminated over the entire surface thereof.

Referring again to FIGS. 4A and 4B, implantable adjunct 300 extends along a length between a proximal end 310 and a distal end 312. Implantable adjunct 300 includes a chamfer 314 at the proximal end 310, although implantable adjunct 300 may have other configurations. In some examples, implantable adjunct 300 can be a foam and/or porous material, such as a thermoset polymer, by way of example only. In additional examples, implantable adjunct 300 can include laminated layers, such as a foam and/or porous material laminated with a mesh material. The laminated layers could include mesh, knit, film, non-woven, or other materials such as healing agents, or coagulating materials, by way of example only. Implantable adjunct 300 includes at least one layer formed of an absorbable material, as known in the art.

Implantable adjunct 300 includes a deck-facing surface 316 and an external surface 318 located on opposing sides thereof. Deck-facing surface 316 and external surface 318 are defined based on the orientation in which implantable adjunct 300 is to be adhered to deck of staple cartridge 100 and are otherwise not intended to be limiting. As described in further detail below, in some examples implantable adjunct 300 can include other laminated layers, such as a mesh material in addition to first film layer 402 and/or second film layer 404 (as shown for example in FIG. 4C). Implantable adjunct 300 also includes a first edge 320 and a second edge 322 located on opposite sides thereof. First edge 320 and second edge 322 both extend along a thickness of implantable adjunct 300 between deck-facing surface 316 and external surface 318.

First film layer 402 includes a film chamfer 406 that is configured to align to chamfer 314 of implantable adjunct 300, although first film layer 402 can have other configurations. First film layer 402 includes a first adjunct facing side 408 and a first exterior side 410 on opposing sides thereof. First adjunct facing side 408 and first exterior side 410 are defined based on the orientation in which first film layer 402 is attached to implantable adjunct 300, as described below, and are otherwise not intended to be limiting. First adjunct facing side 408 contacts deck-facing surface 316 of implantable adjunct 300 when selectively heat laminated thereto, as described in further detail below and illustrated in FIG. 4B, and first exterior side 410 faces away from implantable adjunct 300. In this example, first exterior side 410 can be adhered to deck 108 of staple cartridge 100, for example, using attachment material 302 positioned between first film layer 402 and deck 108, as described above. First film layer 402 can be a thin film layer formed of a thermoplastic material, although first film layer 402 can be formed of other materials. First film layer 402 can be formed in whole or in part of a biodegradable polymer, including but not limited to polymers such as polydioxanone (PDO) or Vicryl, although other flexible absorbable materials could be employed. First film layer 402 in some examples can be a layer that includes a discontinuous first exterior side 410, such as a mesh layer or a knit layer.

First film layer 402 is selectively heat laminated to the deck-facing surface 316 such that there are laminated portions 412 and unlaminated portions 414 of the first film layer 402. First film layer 402 is attached to implantable adjunct 300 at laminated portions 412, where first film layer 402 is melted into implantable adjunct 300. Unlaminated portions 414 provide areas of first film layer 402 that retain the full thickness of first film layer 402, which is advantageous to prevent staple pull through in unlaminated portions 414 during stapling operations. Unlaminated portions 414 further are configured to enable portions of the implantable adjunct 300 adjacent to the unlaminated portions 414 to move independent of the first film layer 402.

The laminated portions 412 are formed by surface laminations 500 formed in first film layer 402. As discussed above, surface laminations 500 are formed from portions of first film layer 402 that are heat melted to implantable adjunct 300 and do not constitute a separate structural element. It is to be understood that surface laminations 500 could be formed in various patterns, including but not limited to the examples set forth herein, to provide the benefits described herein of the selective lamination. The various patterns of surface laminations 500 result in various configurations of laminated portions 412 and unlaminated portions 414 of the first film layer 402.

Referring now specifically to FIGS. 4A and 4B, in this example, surface laminations 500, which provide laminated portions 412, include two lateral rows of lamination 502, two parallel medial rows of lamination 504, and a proximal lamination 506. Lateral rows of lamination 502 are positioned proximate respective side edges 320, 322 of implantable adjunct 300. Medial rows of lamination 504 are positioned proximate a center line of implantable adjunct 300 and extending along longitudinal axis 350 (as shown in FIG. 4A) of implantable adjunct 300. Proximal lamination 506 is positioned proximally and separated from lateral rows of lamination 502 at corner chamfers 314 in implantable adjunct 300. It is to be understood that in other examples surface laminations 500 can include any other number of laminations, including other numbers of lateral, medial, or proximal laminations, located in various configurations on implantable adjunct 300.

In this example, surface laminations 500, including lateral rows of lamination 502, two parallel medial rows of lamination 504, and a proximal lamination 506, are shown as dashed lines having a regular pattern, although other patterns of dashes or dots can be used to form surface laminations 500. The use of dashed lines or dots, for example, for surface laminations 500 allows for flexibility of implantable adjunct 300 between the laminated portions. This helps to prevent lamination tearing or fracturing of implantable adjunct 300, which in turn reduces the number of staples pulled through implantable adjunct 300 during use thereof in a stapling operation.

FIG. 4C is an exploded view of implantable adjunct with an additional film layer 404 that can be adhered to external surface 318 using selective heat lamination to provide laminated portions and unlaminated portions of second film layer 404 similar to those described above with respect to first film layer 402. Second film layer 404 can be a thin film layer formed of a thermoplastic material, although second film layer 404 can be formed of other materials. Second film layer 404 can be formed in whole or in part of a biodegradable polymer, including but not limited to polymers such as polydioxanone (PDO) or Vicryl, although other flexible absorbable materials could be employed. Second film layer 404 in some examples can be a layer that includes a discontinuous exterior side, such as a mesh layer or a knit layer. Second film layer 404 can be provided, for example, to provide improved strength to prevent staple pull through during us of implantable adjunct 300.

The laminated portions for second film layer 404 are formed by surface laminations 507 formed in second film layer 404. As discussed above, surface laminations 507 are formed from portions of second film layer 402 that are heat melted to implantable adjunct 300 and do not constitute a separate structural element. It is to be understood that surface laminations 507 could be formed in various patterns, including but not limited to the examples set forth herein, to provide the benefits described herein of the selective lamination. The various patterns of surface laminations 507 result in various configurations of laminated portions and unlaminated portions of second film layer 404. Although second film layer 404 is illustrated and described with respect to FIG. 4C, it is to be understood that second film layer 404 could be applied to the any of the examples set forth herein in a similar manner.

Referring again to FIG. 4C, in this example, surface laminations 507 are formed in a similar configuration as surface laminations 500 (FIGS. 4A and 4B) and include two lateral rows of lamination 508, two parallel medial rows of lamination 510, and a proximal lamination 512, although in other examples surface laminations 507 could have a different configuration from surface laminations 500.

In this example, surface laminations 507, including lateral rows of lamination 508, two parallel medial rows of lamination 510, and a proximal lamination 512, are similarly shown as dashed lines having a regular pattern, although other patterns of dashes or dots can be used to form surface laminations 507. As described above, the use of dashed lines or dots, for example, for surface laminations 507 allows for flexibility of implantable adjunct 300 between the laminated portions.

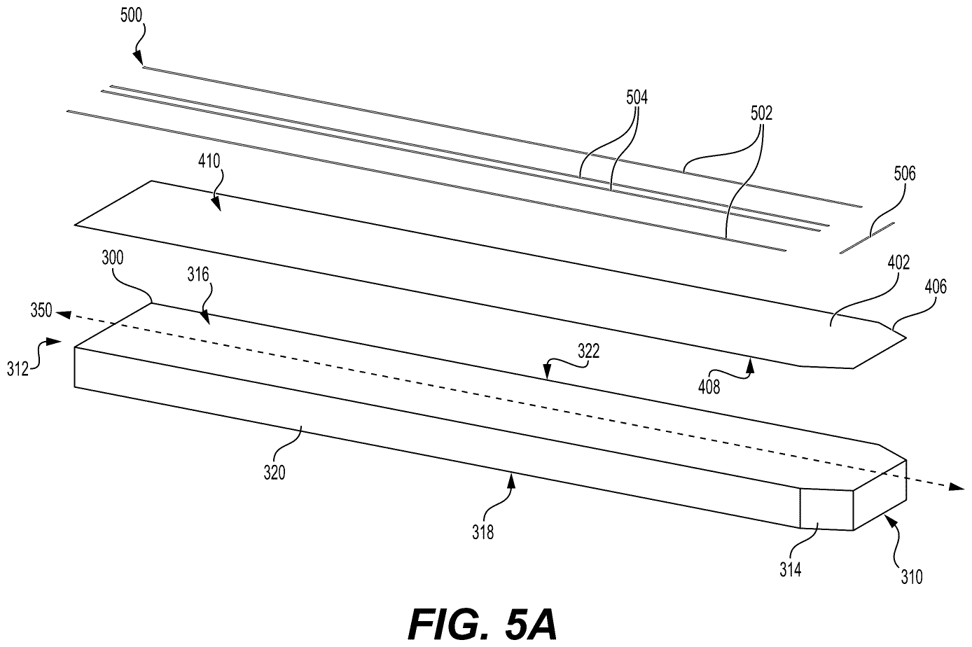

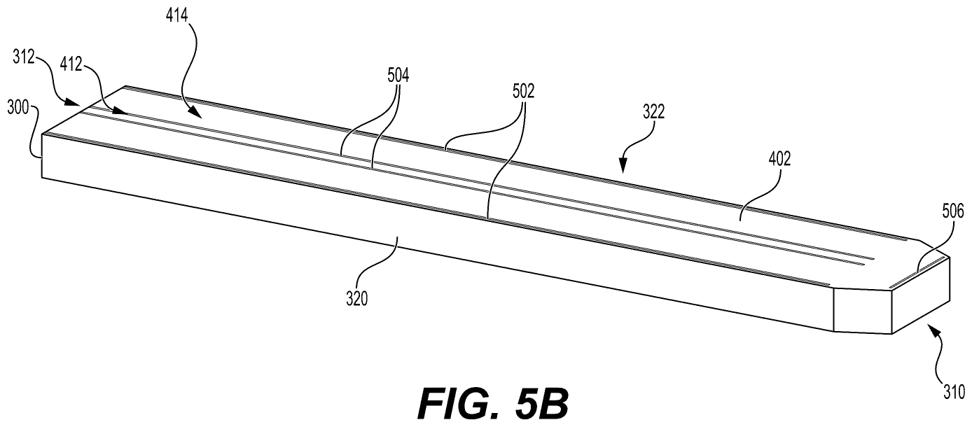

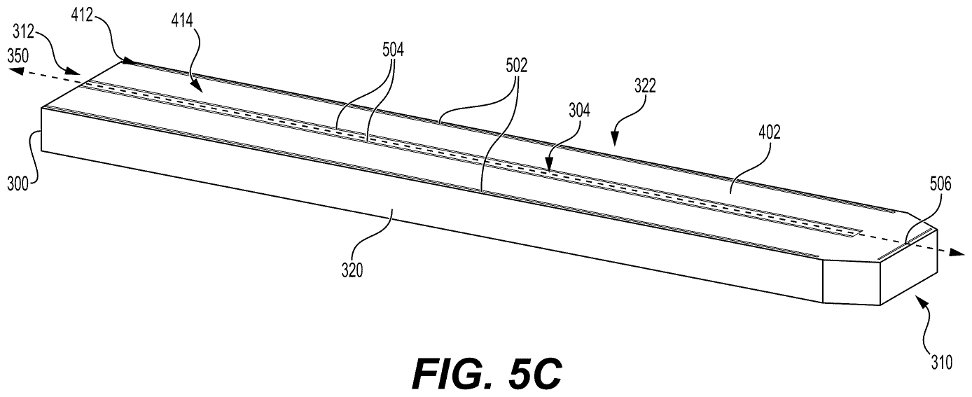

FIGS. 5A and 5B are an exploded view and an assembled view, respectively, of implantable adjunct 300 with first film layer 402 and a lamination pattern formed by surface laminations 500. Implantable adjunct 300 is the same in structure as shown in FIGS. 4A and 4B, expect for as described below. In the example shown in FIGS. 5A and 5B, surface laminations 500, including lateral rows of lamination 502, two parallel medial rows of lamination 504, and a proximal lamination 506, are formed as solid lines of lamination. As described above, surface laminations 500 are formed from portions of first film layer 402 that are heat melted to implantable adjunct 300 and do not constitute a separate structural element. It is to be understood that second film layer 404 (as shown in FIG. 4C) could similarly be selectively laminated to the opposite side of implantable adjunct 300 using surface laminations 500 shown in FIGS. 5A and 5B, although other surface lamination patterns could be employed to selectively lamination second film layer 404.

FIG. 5C is a perspective view of an implantable adjunct 300 similar to implantable adjunct 300 shown in FIGS. 5A and 5B. In the example of FIG. 5C, implantable adjunct 300 further includes a groove 304 located along longitudinal axis 350 of implantable adjunct. Groove 304 is located between medial rows of lamination 504 and extends at least partially through a thickness of implantable adjunct 300. Groove 304 can be formed, for example, as described in further detail below, using a higher-powered laser setting than what is used during the heat lamination process. Groove 304, for example, can be formed by completely melting first film layer 402 in order to perforate or separate the two halves of implantable adjunct 300 along groove 304. Groove 304 can, for example, provide easier or cleaner cutline performance during use of implantable adjunct 300.

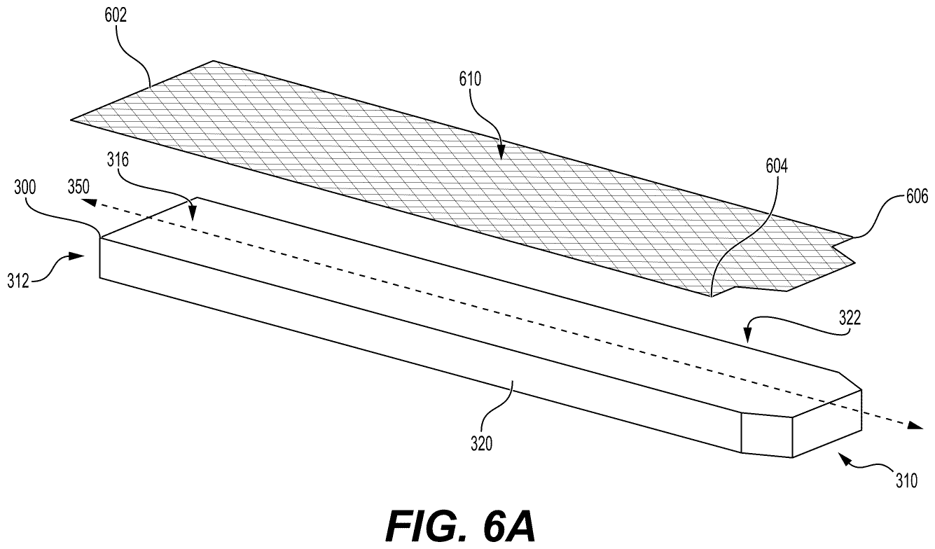

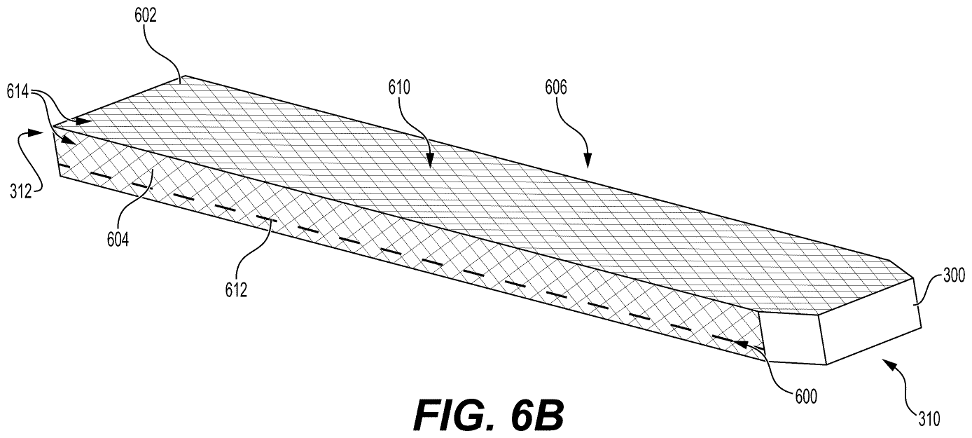

FIGS. 6A and 6B are an exploded view and an assembled view, respectively, of another exemplary implantable adjunct 300 configured to be detachably adhered to deck 108 of staple cartridge 100. Implantable adjunct 300 can be employed in the system described above with respect to FIGS. 2A-2C, for example. Referring again to FIGS. 6A and 6B, implantable adjunct 300 includes a first film layer 602 selectively heat laminated thereto using side laminations 600, as described in further detail below. Side laminations 600 are formed by portions of first film layer 602 that are heat melted, in this example, to edges 320 and 322 of implantable adjunct 300 using known heat lamination processes in order to attach first film layer 402 to implantable adjunct 300. As discussed above, selective lamination provides a number of advantages. The use of side laminations 600 further enables the staple contacting film on deck-facing surface 316 of implantable adjunct 300 to better flex and stretch, during use of implantable adjunct 300, to prevent film tearing or fracturing around the staple crowns, and thus reduces staple pull-through. The use of side laminations 600 further prevents delamination of the film layer as a result of tissue manipulation shear forces during use of implantable adjunct 300 and prevents early release of implantable adjunct 300 via moisture received through any possible micro-holes in the film layer that may be formed during lamination.

Referring again to FIGS. 6A and 6B, implantable adjunct 300 is the same in structure as described above. First film layer 602 is similar in structure to first film layer 402 except as described below. In this example, first film layer 602 includes first wings 604 and second wings 606 provided for side lamination, as described in further detail below. First wing 604 and second wing 606 extend laterally and are configured to at least partially covering first edge 320 and second edge 322 of the implantable adjunct 300 when first film layer 602 is in contact with deck-facing surface 316 such that first exterior side 610 facing away from implantable adjunct 300. First wing 604 and second wing 606 can be folded over into the position shown in FIG. 6B prior to lamination.

Referring now more specifically to FIG. 6B, first film layer 602 is selectively heat laminated to implantable adjunct 300 such that there are laminated portions 612 and unlaminated portions 614 of the first film layer 602. First film layer 602 is attached to implantable adjunct 300 at laminated portions 612, which in this example are located along first edge 320 and second edge 322 of implantable adjunct 300. Laminated portions 612 are formed in areas where first film layer 602 is melted into implantable adjunct 300. Unlaminated portions 614 provide areas of first film layer 602 that retain the full thickness of first film layer 602, which is advantageous to prevent staple pull through in unlaminated portions 614 during stapling operations. Unlaminated portions 414 further are configured to enable portions of the implantable adjunct 300 adjacent to the unlaminated portions 614 to move independent of the first film layer 602. In this example, the entire first exterior side 610 of first film layer 602, which contacts deck-facing surface 316, if fully unlaminated, i.e., part of unlaminated portions 414.

The laminated portions 612 are formed by side laminations 600 formed in first film layer 602 in first and second wings 604, 606 along first edge 320 and second edge 322, respectively. Side laminations 600 (similar to surface laminations 500 described above) are formed from portions of first film layer 602 that are heat melted to implantable adjunct 300 and do not constitute a separate structural element. It is to be understood that side laminations 600 could be formed in various patterns. Side laminations 600, in this example, are positioned along first wing 604 and second wing 606, thereby connecting first film layer 602 to respective edges 320, 322 of implantable adjunct 300.

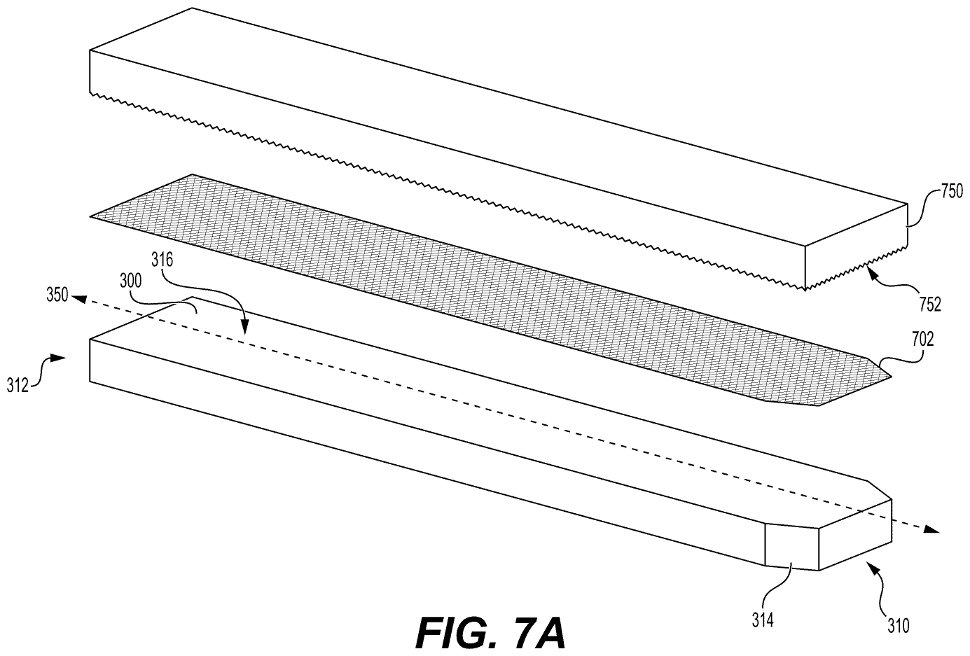

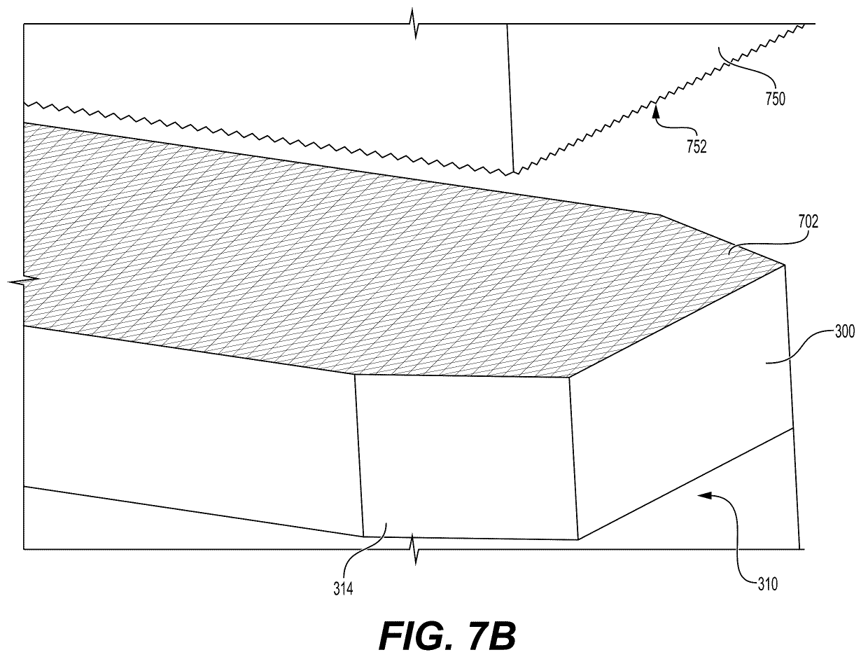

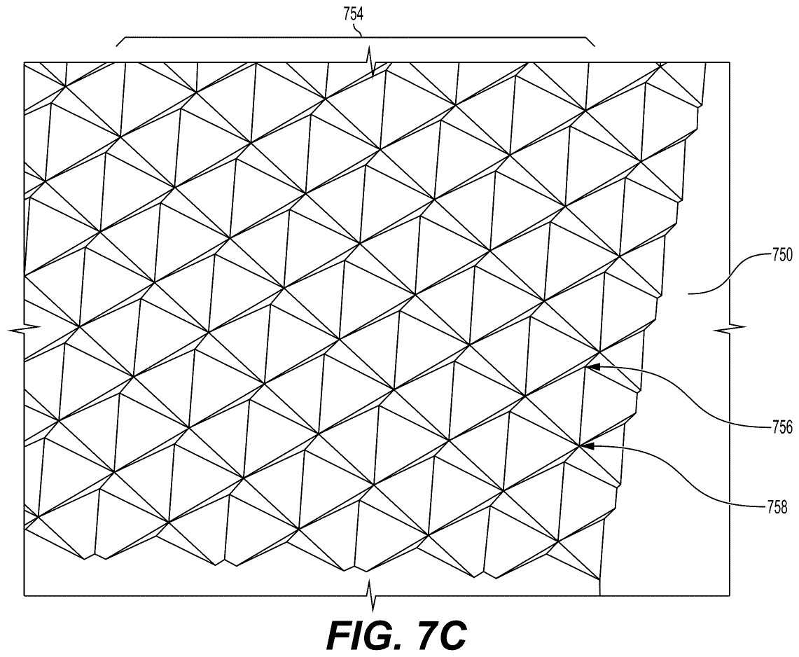

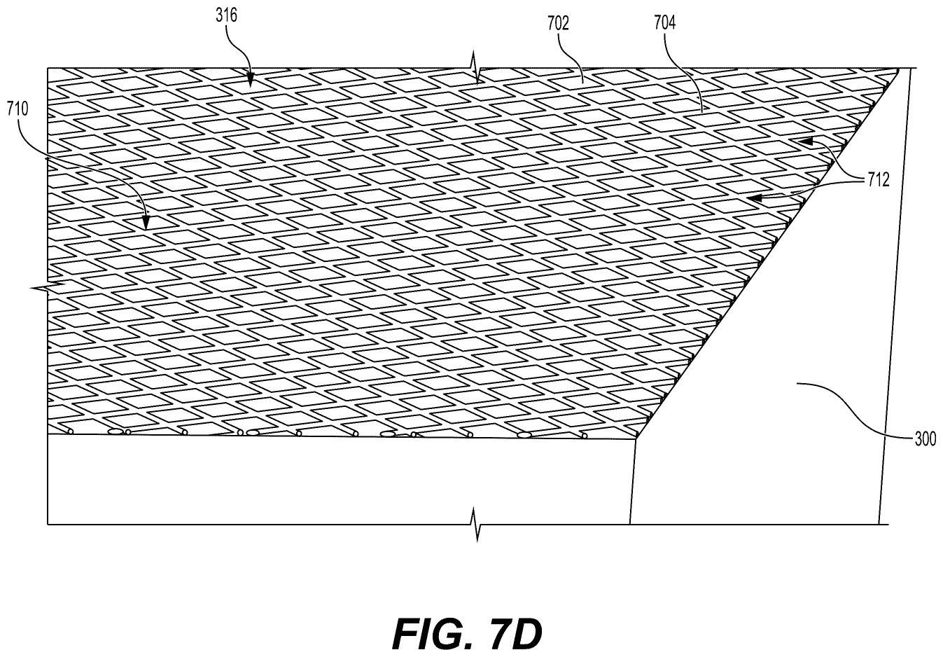

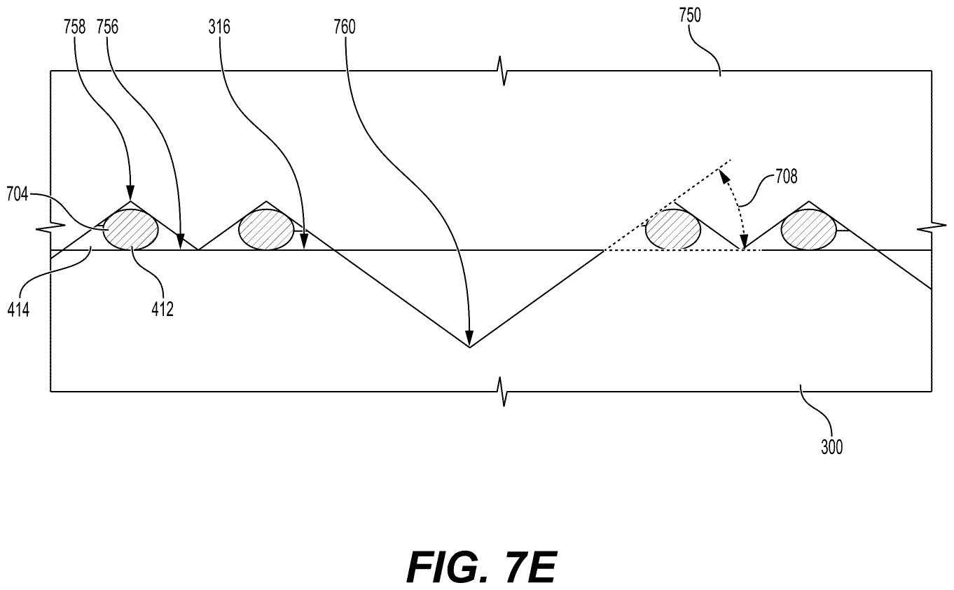

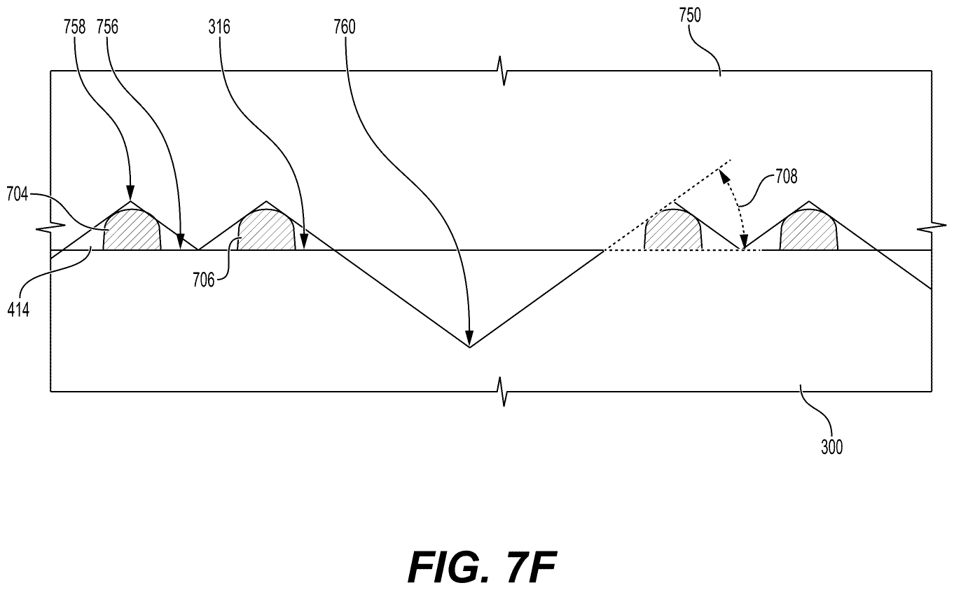

FIG. 7A illustrates a system for laminating a first film layer 702 formed of a mesh material or a knit fabric to an implantable adjunct 300 using a lamination block 750. Lamination block 750 is used to laminate first film layer 702 to implantable adjunct 300. Lamination block 750 includes a lamination surface 752 that is used to selectively laminate first film layer 702 to implantable adjunct 300, as described in further detail below, by pressing lamination surface 752 into contact with first film layer 702 and implantable adjunct 300 using known heat lamination techniques. In other examples, a heat source could be applied to the side of first film layer 702 to be adhered to implantable adjunct 300 just before the two materials are pressed together. In this example, lamination block 750 holds first film layer 702 in place and provides a force on first film layer 702. Although first film layer 702 is described as being heat laminated, in other examples, first film layer 702 could be adhered to implantable adjunct 300 using a bonding adhesive or through a separate bonding layer.

In this example, implantable adjunct 300 is the same in structure as described above. First film layer 702 is similar in structure to first film layer 402 except as described below. In this example, first film layer 702 is formed of a mesh material having a plurality of threads or strands 704 (as shown in FIG. 7D) that can be formed in whole or in part of a biodegradable polymer, including but not limited to polymers such as polydioxanone (PDO) or Vicryl, although other flexible absorbable materials could be employed. In some examples, first film layer 702 has a thickness of about 1 micron to about 5 microns, although other thicknesses can be employed.

Lamination block 750 includes a lamination surface 752 that is used to selectively laminate first film layer 702 to implantable adjunct 300, as described in further detail below. FIG. 7B illustrates the system of FIG. 7A with first film layer 702 selectively laminated to deck-facing surface 316 of implantable adjunct 300 forming a weave pattern, as described in further detail below.

FIG. 7C is a detail view of a lamination surface texture pattern 754 on lamination surface 752 of lamination block 750. In this example, lamination surface texture pattern 754 includes a number of peaks 756 and troughs 758 that interact with the mesh first film layer 702 as described below to form a weave pattern that selectively laminates first film layer 702 to implantable adjunct 300, as described below. Lamination surface texture pattern 754 may have other elements in other combinations to selectively laminate first film layer 702 to implantable adjunct 300 in accordance with the disclosure herein.

FIG. 7D is a detail view of implantable adjunct 300 with first film layer 702 selectively laminated thereon. First film layer 702 is selectively heat laminated to deck-facing surface 316 of implantable adjunct by contact with lamination surface texture pattern 754 (shown in FIG. 7C) of lamination block 750, such that there are laminated portions 712 of first film layer 702. First film layer 702 includes fiber strands 704 that are formed into a weave pattern in laminated portions 712, which attach the first film layer 702 to the deck-facing surface 316 of the implantable adjunct 300. The shape of the weave pattern of fiber strands 704 corresponds to lamination surface texture pattern 754 of a lamination block 750 that formed the weave pattern.

FIG. 7E is cross-sectional view of the system of FIG. 7A being used to laminate the mesh first film layer 702 comprising fiber strands 704 to implantable adjunct 300 using lamination block 850. Fiber strands 704 are located in troughs 758 when peaks 756 contact implantable adjunct 300 and intermediate peaks 760 are compressed into implantable adjunct 300. A pitch 708 of fiber strands 704 matches the lamination surface texture pattern 754 of lamination surface 752. FIG. 7F is cross-sectional view of the system of FIG. 7A being used to laminate the mesh first film layer. Fiber strands 704 are formed with flattened profiles 706 when laminated to implantable adjunct 300.

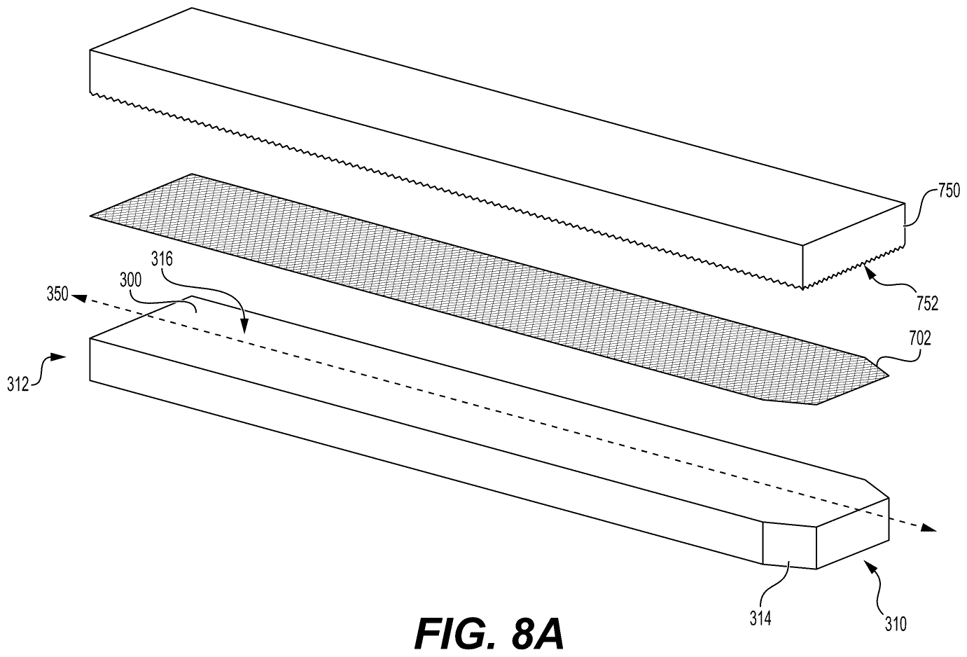

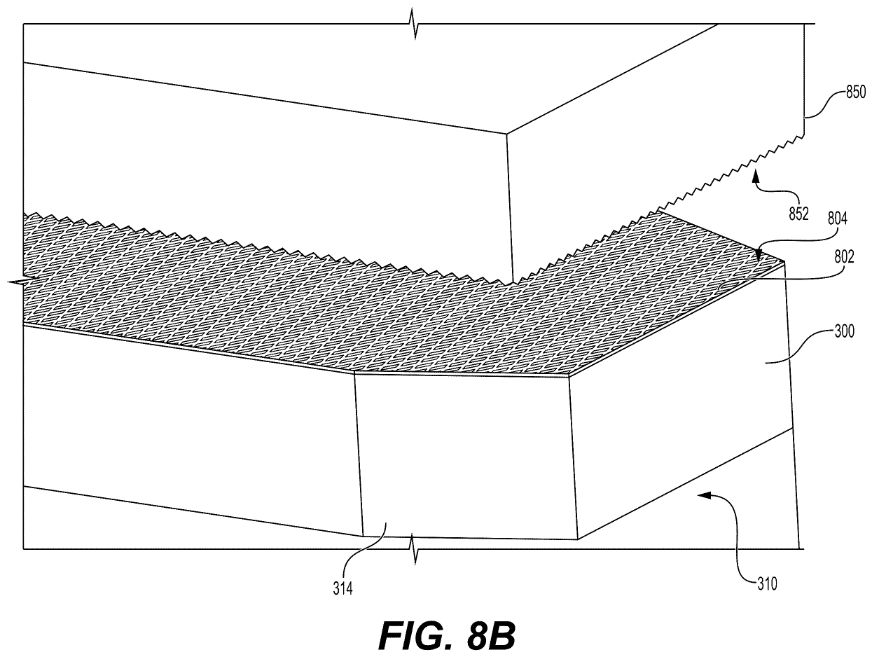

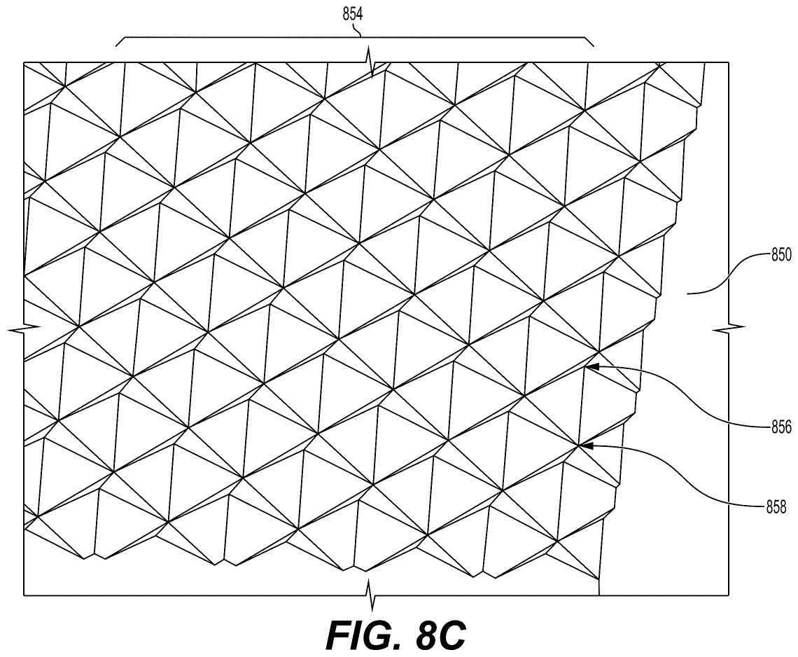

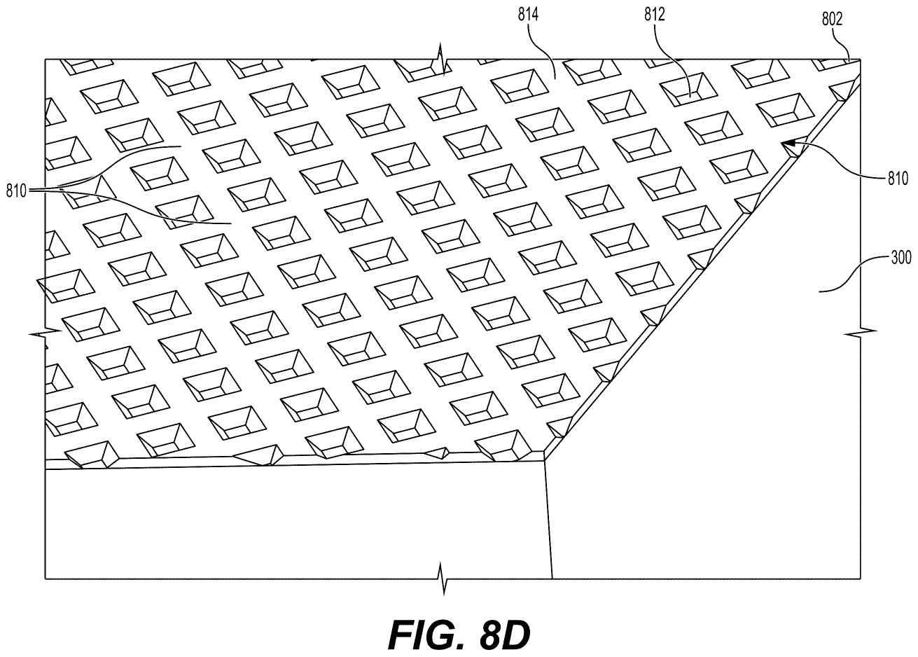

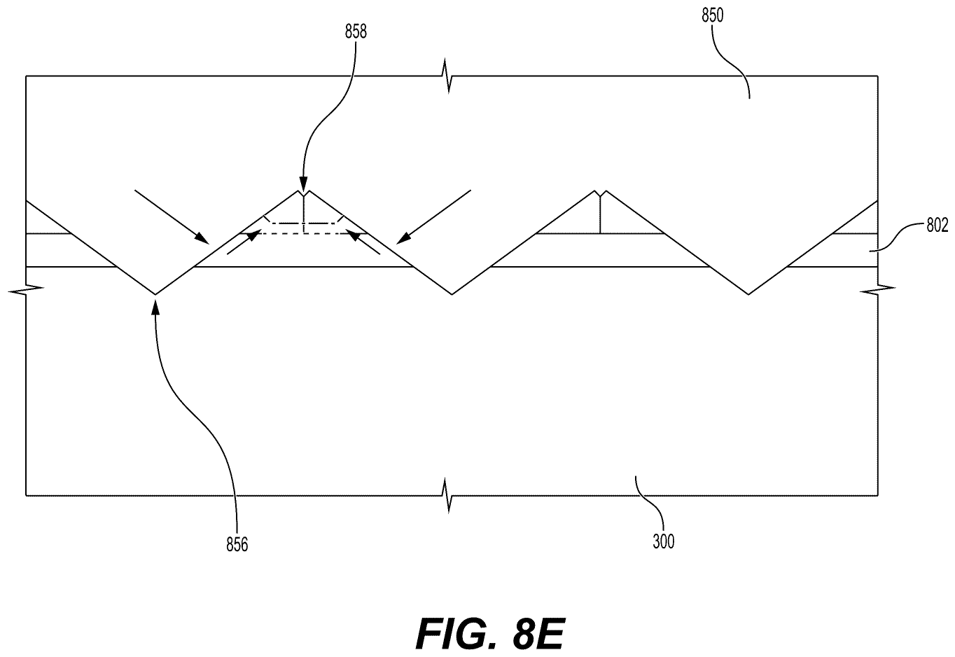

FIG. 8A illustrates another system for laminating a first film layer 802 formed of a solid film material to an implantable adjunct 300 using a lamination block 850. Lamination block 850 is used to selectively laminate first film layer 802 to implantable adjunct 300. Lamination block 850 includes a lamination surface 852 that is used to selectively laminate first film layer 802 to implantable adjunct 300, as described in further detail below, by pressing lamination surface 852 into contact with first film layer 802 and implantable adjunct 300 using known heat lamination techniques. Lamination surface 852 can be provided with an anti-stick coating, such as Teflon or ceramic, to prevent film layer 802 from sticking thereto, although other hard anodized materials could be used.

In this example, implantable adjunct 300 is the same in structure as described above. First film layer 802 is similar in structure to first film layer 402 except as described below. In this example, first film layer 802 is formed of a solid film material that can be formed in whole or in part of a biodegradable polymer, including but not limited to polymers such as polydioxanone (PDO) or Vicryl, although other flexible absorbable materials could be employed. In some examples, first film layer 802 has a thickness of about 1 micron to about 2 microns, although other thicknesses can be employed.

Lamination block 850 includes a lamination surface 852 that is used to selectively laminate first film layer 802 to implantable adjunct 300, as described in further detail below. FIG. 8B illustrates the system of FIG. 8A with first film layer 802 selectively laminated to deck-facing surface 316 of implantable adjunct 300, as described in further detail below. In this example, first film layer 802 includes surface laminations 804 formed thereon as a result of interaction with lamination surface 852.

FIG. 8C is a detail view of a lamination surface texture pattern 854 on lamination surface 852 of lamination block 850. In this example, lamination surface texture pattern 854 includes a number of peaks 856 and troughs 858 that interact with first film layer 802 as described below to form surface laminations 804. Surface laminations 804 are areas where first film layer 802 is melted into implantable adjunct 300 and do not provide additional structural elements. Laminations surface texture pattern 854 selectively laminates first film layer 802 to implantable adjunct 300, as described below. Lamination surface texture pattern 854 may have other elements in other combinations to selectively laminate first film layer 802 to implantable adjunct 300 in accordance with the disclosure herein.

FIG. 8D is a detail view of implantable adjunct 300 with first film layer 802 selectively laminated thereon. First film layer 802 is selectively heat laminated to deck-facing surface 316 of implantable adjunct by contact with lamination surface texture pattern 854 (shown in FIG. 8C) of lamination block 850 on exterior side 810. The contact forms laminated portions 812 and unlaminated portions 814 of first film layer 802. Laminated portions 812 are areas of first film layer 802 melted into implantable adjunct 300 that attach first film layer 802 to the deck-facing surface 316 of implantable adjunct 300. Unlaminated portions 814 are configured to enable portions of the implantable adjunct 300 adjacent to unlaminated portions 714 to move independent of the first film layer 702.

FIG. 8E is cross-sectional view of the system of FIG. 8A being used to laminate first film layer 802 to implantable adjunct 300 using lamination block 850. Peaks 856 melt first film layer 802 to form surface laminations 804 (as shown in FIG. 8B), which are holes in first film layer 802 where the material of first film layer 802 is melted or bonded to implantable adjunct 300. As a result, first film layer 802 becomes a mesh connected to implantable adjunct 300. The mesh layer formed from first film layer 802 has increased thickness as melted portions are pushed outward to form struts that increase the strength of first film layer 802 in those areas near troughs 858 of lamination block 850. In some examples, lamination block 850 could further be formed to create a groove, such as groove 304 shown in FIG. 5C, by way of example.

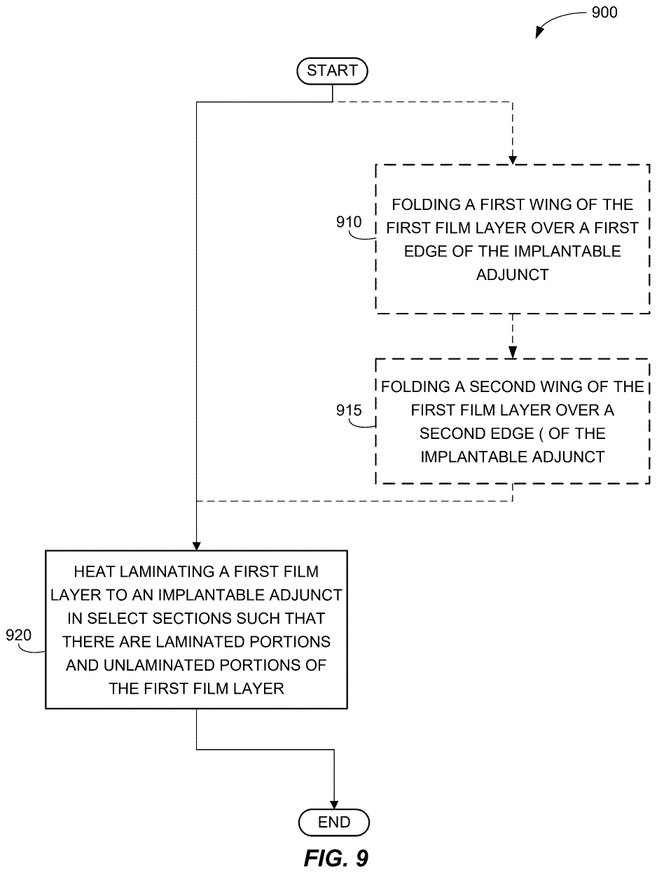

FIG. 9 is a flowchart of a method 900 for assembling an implantable adjunct 300. Method 900 will now be described with reference to FIGS. 1-9. In optional step 910, when using first film layer 602 to form side laminations 600 (as shown in FIGS. 6A and 6B), first film layer 602 is folded over first edge 320 of the implantable adjunct 300. In optional step 915, second wing 606 of the first film layer 602 is then folded over second edge 322 of the implantable adjunct 300.

Next, in step 920, a first film layer, such as any of the first film layers 402, 602, 702, 802 described herein, is heat laminated to implantable adjunct 300 in select sections such that there are laminated portions (e.g., laminated portions 412, 612, 712, 812) and unlaminated portions (e.g., unlaminated portions 414, 614, 814).

In one example, the localized laminations could be applied using a laser at a first intensity to direct heat only to the localized areas. A thin layer of Teflon PFTE sheet can be used to apply pressure to the film layer as the laser heat passes through the Teflon PFTE sheet and laminates only the desired areas. Many other methods could be used to apply localized lamination zones to selectively laminate film layer to implantable adjunct.

In one example, the heat laminating of step 920 includes forming surface laminations 500 in longitudinal rows 502, 504 along first film layer 402 (as shown in FIGS. 4A-4C, 5A and 5B), although the heat lamination may be formed in other configurations, such as proximal lamination 506 for example. The heat lamination attaches the first film layer 402 to deck-facing surface 316 of the implantable adjunct 300. In one example, longitudinal rows 502, 504 include two parallel medial rows of lamination 504. In some examples, the method further includes using the laser at a second intensity that is greater than the first intensity to cut groove 304 between the parallel medial rows of lamination 504 (as shown in FIG. 5C). Groove 304 is formed by more completely melting the film layer in order to perforate or separate the two halves of implantable adjunct 300, allowing for easier or cleaner cutline performance.

In another example, the heat laminating of step 920 includes forming side laminations 600 along first wing 604 and second wing 606.

In a further example, the heat laminating of step 920 includes using lamination block 750 (FIG. 7A) or lamination block 850 (FIG. 8A) to form a pattern of the laminated portions 712 (FIG. 7D) or 812 (FIG. 8D) corresponding to the surface texture pattern 754 (FIG. 7C), 854 (FIG. 8C) of the lamination block 750, 850. For example, the laminated portions 712, 812 correspond to locations of peaks 756, 856, respectively, in the surface texture pattern 754, 854. In the example of FIG. 8D, unlaminated portions 814 are formed by the location of troughs 858 in surface texture pattern 854.

Examples of the present disclosure can be implemented by any of the following numbered clauses:
Clause 1: An implantable adjunct (300) configured to be detachably adhered to a deck (108) of a staple cartridge (100), the implantable adjunct (300) comprising: a deck-facing surface (316); an external surface (318); and a first film layer (402, 602, 702, 802) contacting the deck-facing surface (316), the first film layer (402, 602, 702, 802) comprising a first exterior side (410, 610, 710, 810) facing away from the implantable adjunct (300), wherein the first film layer (402, 602, 702, 802) is selectively heat laminated to the deck-facing surface (316) such that there are laminated portions (412, 612, 712, 812) and unlaminated portions (414, 614, 814) of the first film layer (402, 602, 702, 802).
Clause 2: The implantable adjunct (300) of Clause 1, wherein the laminated portions (412) are surface laminations (500) positioned along the first exterior side (410), the surface laminations (500) comprise two lateral rows of lamination (502), each of the two lateral rows of lamination (502) positioned proximate respective side edges (320, 322) of the implantable adjunct (300).
Clause 3: The implantable adjunct (300) of Clause 1 or 2, wherein the laminated portions (412) comprise one or more medial rows of lamination (504) positioned proximate a center line of the implantable adjunct (300) and extending along a longitudinal axis (350) of the implantable adjunct (300).
Clause 4: The implantable adjunct (300) of Clause 3, comprising two parallel medial rows of lamination (504), and further comprising a groove (304) positioned between the parallel medial rows of lamination (504), the groove (304) extending at least partially through a thickness of the implantable adjunct (300).
Clause 5: The implantable adjunct (300) of any one of Clauses 2 to 4, wherein the two lateral rows of lamination (502) are dashed lines of surface laminations (500).
Clause 6: The implantable adjunct (300) of any one of Clauses 2 to 5 further comprising a proximal lamination (506) positioned proximally and separated from the two lateral rows of lamination (502) at corner chamfers (314) in the implantable adjunct (300).
Clause 7: The implantable adjunct (300) of any one of the preceding clauses further comprising a second film layer (404) selectively heat laminated to the external surface (318) such that there are laminated portions and unlaminated portions of the second film layer (402).
Clause 8: The implantable adjunct (300) of Clause 1, wherein the first film layer (602) comprises a first wing (604) and a second wing (606) extending laterally, the first wing (604) at least partially covering a first edge (320) of the implantable adjunct (300), and the second wing (606) at least partially covering a second edge (322) of the implantable adjunct (300).
Clause 9: The implantable adjunct (300) of Clause 8, wherein the laminated portions (612) are side laminations (600) positioned along the first wing (604) and the second wing (606), thereby connecting the first film layer (602) to the edges (320, 322) of the implantable adjunct (300).
Clause 10: The implantable adjunct (300) of Clause 8 or 9, wherein the first exterior side (610) is fully unlaminated.
Clause 11: The implantable adjunct (300) of Clause 1, wherein the laminated portions (712, 812) are formed into a weave pattern and attach the first film layer (702, 802) to the deck-facing surface (316) of the implantable adjunct (300).
Clause 12: The implantable adjunct (300) of Clause 11, wherein a shape of the weave pattern corresponds to a lamination surface texture pattern (754, 854) of a lamination block (750, 850) that formed the weave pattern.
Clause 13: The implantable adjunct (300) of Clause 11, wherein the first film layer (702) is a mesh material comprising a plurality of fiber strands (704) forming the weave pattern.
Clause 14: The implantable adjunct (300) of any of the preceding clauses, wherein the unlaminated portions (414, 614, 714, 814) are configured to enable portions of the implantable adjunct (300) adjacent to the unlaminated portions (414) to move independent of the first film layer (402, 602, 702, 802).
Clause 15: The implantable adjunct (300) of any one of the preceding clauses, further comprising the staple cartridge (100), wherein the implantable adjunct (300) is adhered to the deck (108) by an attachment material (302) positioned between the first film layer (402) and the deck (108).
Clause 16: A method for assembling an implantable adjunct (300), the method comprising: heat laminating a first film layer (402, 602, 702, 802) to the implantable adjunct (300) in select sections such that there are laminated portions (412, 612, 712, 812) and unlaminated portions (414, 614, 814) of the first film layer (402, 602, 702, 802), the laminated portions (412, 612, 712, 812) attaching the first film layer (402, 602, 702, 802) to the implantable adjunct (300).
Clause 17: The method of Clause 16, wherein heat laminating comprises forming surface laminations (500) in longitudinal rows (502, 504) along the first film layer (402), and wherein heat lamination attaches the first film layer (402) to a deck-facing surface (316) of the implantable adjunct (300).
Clause 18: The method of Clause 17, wherein: the longitudinal rows (502, 504) comprise two parallel medial rows of lamination (504); heat laminating comprises using a laser set at a first intensity to form the surface laminations (500); and the method further comprises using the laser at a second intensity to cut a groove (304) between the parallel medial rows of lamination (504), the second intensity being greater than the first intensity.
Clause 19: The method of Clause 16 further comprising: folding a first wing (604) of the first film layer (602) over a first edge (320) of the implantable adjunct (300); and folding a second wing (606) of the first film layer (602) over a second edge (322) of the implantable adjunct (300), wherein heat laminating comprises laminating first wing (604) to the first edge (320) and laminating the second wing (606) to the second edge (322).
Clause 20: The method of Clause 16, wherein: heat laminating comprises using a lamination block (750, 850) to form the laminated portions (712, 812) and unlaminated portions (414, 614, 814), a pattern of the laminated portions (712, 812) corresponding to a surface texture pattern (754, 854) of the lamination block (750, 850); the laminated portions (712, 812) correspond to locations of peaks (756, 856) in the surface texture pattern (754, 854); and the unlaminated portions (414, 614, 814) correspond to locations of troughs (758, 858) in the surface texture pattern (754, 854).
Clause 21: The method of Clause 20, wherein the first film layer (702) is a mesh material comprising a plurality of fiber strands (704) forming a weave pattern.

In describing example embodiments, terminology has been resorted to for the sake of clarity. As a result, not all possible combinations have been listed, and such variants are often apparent to those of skill in the art and are intended to be within the scope of the claims which follow. It is intended that each term contemplates its broadest meaning as understood by those skilled in the art and includes all technical equivalents that operate in a similar manner to accomplish a similar purpose without departing from the scope and spirit of the invention. It is also to be understood that the mention of one or more steps of a method does not preclude the presence of additional method steps or intervening method steps between those steps expressly identified. Similarly, some steps of a method can be performed in a different order than those described herein without departing from the scope of the disclosed technology.

1-15. (canceled)

16. An implantable adjunct configured to be detachably adhered to a deck of a staple cartridge, the implantable adjunct comprising:
a deck-facing surface;
an external surface; and
a first film layer contacting the deck-facing surface, the first film layer comprising a first exterior side facing away from the implantable adjunct,
wherein the first film layer is selectively heat laminated to the deck-facing surface such that there are laminated portions and unlaminated portions of the first film layer.

17. The implantable adjunct of claim 16, wherein the laminated portions are surface laminations positioned along the first exterior side, the surface laminations comprise two lateral rows of lamination, each of the two lateral rows of lamination positioned proximate respective side edges of the implantable adjunct.

18. The implantable adjunct of claim 16, wherein the laminated portions comprise one or more medial rows of lamination positioned proximate a center line of the implantable adjunct and extending along a longitudinal axis of the implantable adjunct.

19. The implantable adjunct of claim 18, comprising two parallel medial rows of lamination, and further comprising a groove positioned between the parallel medial rows of lamination, the groove extending at least partially through a thickness of the implantable adjunct.

20. The implantable adjunct of claim 17, wherein the two lateral rows of lamination are dashed lines of surface laminations.

21. The implantable adjunct of claim 17 further comprising a proximal lamination positioned proximally and separated from the two lateral rows of lamination at corner chamfers in the implantable adjunct.

22. The implantable adjunct of claim 16 further comprising a second film layer selectively heat laminated to the external surface such that there are laminated portions and unlaminated portions of the second film layer.

23. The implantable adjunct of claim 16, wherein the first film layer comprises a first wing and a second wing extending laterally, the first wing at least partially covering a first edge of the implantable adjunct, and the second wing at least partially covering a second edge of the implantable adjunct.

24. The implantable adjunct of claim 23, wherein the laminated portions are side laminations positioned along the first wing and the second wing, thereby connecting the first film layer to the edges of the implantable adjunct.

25. The implantable adjunct of claim 23, wherein the first exterior side is fully unlaminated.

26. The implantable adjunct of claim 16, wherein the laminated portions are formed into a weave pattern and attach the first film layer to the deck-facing surface of the implantable adjunct.

27. The implantable adjunct of claim 26, wherein a shape of the weave pattern corresponds to a lamination surface texture pattern of a lamination block that formed the weave pattern.

28. The implantable adjunct of claim 26, wherein the first film layer is a mesh material comprising a plurality of fiber strands forming the weave pattern.

29. The implantable adjunct of claim 16, wherein the unlaminated portions are configured to enable portions of the implantable adjunct adjacent to the unlaminated portions to move independent of the first film layer.

30. The implantable adjunct of claim 16, further comprising the staple cartridge, wherein the implantable adjunct is adhered to the deck by an attachment material positioned between the first film layer and the deck.

31. A method for assembling an implantable adjunct, the method comprising:
heat laminating a first film layer to the implantable adjunct in select sections such that there are laminated portions and unlaminated portions of the first film layer, the laminated portions attaching the first film layer to the implantable adjunct.

32. The method of claim 31, wherein heat laminating comprises forming surface laminations in longitudinal rows along the first film layer, and wherein heat lamination attaches the first film layer to a deck-facing surface of the implantable adjunct.

33. The method of claim 32, wherein:
the longitudinal rows comprise two parallel medial rows of lamination;
heat laminating comprises using a laser set at a first intensity to form the surface laminations; and
the method further comprises using the laser at a second intensity to cut a groove between the parallel medial rows of lamination, the second intensity being greater than the first intensity.

34. The method of claim 31 further comprising:
folding a first wing of the first film layer over a first edge of the implantable adjunct; and
folding a second wing of the first film layer over a second edge of the implantable adjunct,
wherein heat laminating comprises laminating first wing to the first edge and laminating the second wing to the second edge.

35. The method of claim 31, wherein:
heat laminating comprises using a lamination block to form the laminated portions and unlaminated portions, a pattern of the laminated portions corresponding to a surface texture pattern of the lamination block;
the laminated portions correspond to locations of peaks in the surface texture pattern; and
the unlaminated portions correspond to locations of troughs in the surface texture pattern.
